(19) United States
(12) Patent Application Publication
(10) Pub. No.: US 2019/0282561 A1
(43) Pub. Date: Sep. 19, 2019

(54) Combinations of Glycopyrrolate and Beta2 Adrenoceptor Agonists

(71) Applicant: Novartis AG, Basel (CH)

(72) Inventor: Stephen Paul Collingwood, Horsham (GB)

(21) Appl. No.: 16/429,382

(22) Filed: Jun. 3, 2019

Related U.S. Application Data
(63) Continuation of application No. 15/818,991, filed on Nov. 21, 2017, now abandoned.
Application No. 15/818,991 is a continuation of application No. 14/973,778, filed on Dec. 18, 2015, now abandoned.
Application No. 14/973,778 is a continuation of application No. 13/862,529, filed on Apr. 15, 2013, now abandoned.
Application No. 13/862,529 is a continuation of application No. 11/568,559, filed on Jul. 7, 2008, now abandoned.

(30) Foreign Application Priority Data
May 18, 2004 (GB) 0411056.5

(51) Int. Cl. A61K 31/4704; A61K 31/4015; A61K 31/402; A61K 31/40

(52) U.S. Cl. CPC A61K 31/4704; A61K 31/40; A61K 31/402; A61K 31/4015

(57) ABSTRACT

A medicament comprising, separately or together (A) glycopyrrolate; and (B) either a compound of formula

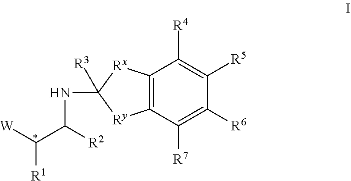

in free or salt or solvate form, wherein W, R^{X}, R^{Y}, R¹, R², R³, R⁴, R⁵, R⁶ and R⁷ have the meanings as indicated in the specification, or a compound of formula II

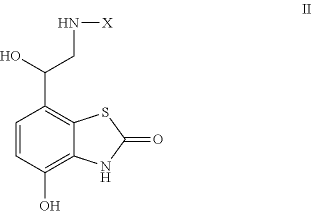

in free or salt or solvate form, wherein X has the meaning as indicated in the specification, for simultaneous, sequential or separate administration in the treatment of an inflammatory or obstructive airways disease. Pharmaceutical compositions that contain (A) and (B) are also described.

This invention relates to organic compounds and their use as pharmaceuticals, in particular for the treatment of inflammatory or obstructive airways diseases.

In one aspect, the present invention provides a medicament comprising, separately or together
(A) glycopyrrolate; and
(B) either a compound of formula I

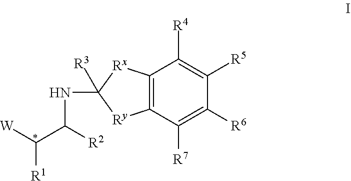

In free or salt or solvate form, wherein
W is a group of formula

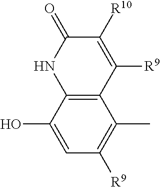

R^{x} and R⁷ are both —CH₁— or —(CH₂)₂—:
R¹ is hydrogen, hydroxy, or C₁-C₁₀-alkoxy;
R² and R³ are each independently hydrogen or C₁-C₁₀-alkyl;
R⁴, R⁵, R⁶ and R⁷ are each independently hydrogen, halogen, cyano, hydroxy, C₁-C₁₀-alkoxy, C₆-C₁₀-aryl, C₁-C₁₀-alkyl, C₁-C₁₀-alkyl substituted by one or more halogen atoms or one or more hydroxy or C₁-C₁₀-alkoxy groups, C₁,-C₁₀-alkyl interrupted by one or more hetero atoms, C₂-C₁₀-alkenyl, trialkylsilyl, carboxy, C₁-C₁₀-alkoxycarbonyl, or —CONR¹¹R¹² where R¹¹ and R¹² are each independently hydrogen or C₁-C₁₀-alkyl,
or R⁴ and R⁵, R⁵ and R⁶, or R⁶ and R⁷ together with the carbon atoms to which they are attached denote a 5-, 6- or 7-membered carbocyclic ring or a 4- to 10-membered heterocyclic ring; and
R⁸, R⁹ and R¹⁰ are each independently hydrogen or C₁-C₄-alkyl;
or a compound of formula II

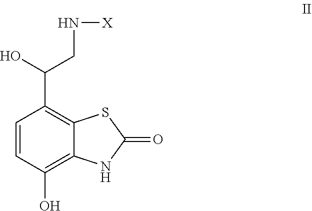

in free or sale or solvate form, wherein
X is —R¹³—Ar—R¹⁴ or —R¹⁵—Y;
Ar denotes a phenylene group optionally substituted by halo, hydroxy, C₁-C₁₀-alkyl, C₁-C₁₆-alkoxy, C₁-C₁₀-alkoxy-C₁-C-alkyl, phenyl, C₁-C₁₀-alkyl substituted by phenyl, C₁-C₁₀-alkoxy substituted by phenyl, C₁-C₁₀-alkyl-substituted phenyl or by C₁-C₁₀-alkoxy-substituted phenyl;
R¹³ and R¹⁴ are attached to adjacent carbon atoms in Ar, and
either R¹³ is C₁C₁₀-alkylene and R¹⁴ is hydrogen, C₁-C₁₀-alkyl, C₁-C₁₀-alkoxy or halogen,
or R¹⁵ and R¹⁴ together with the carbon atoms in Ar to which they are attached denote a 5-, 6- or 7-membered cycloaliphatic ring;
R¹⁵ is a bond or C₁-C₁₀-alkylene optionally substituted by hydroxy, C₁-C₁₀-alkoxy, C₆-C₁₀-aryl or C₇-C₁₄-aralkyl; and
Y is C₁-C₁₀-alkyl, C₁-C₁₀-alkoxy, C₂-C₁₀-alkenyl or C₂-C₁₀-alkynyl optionally substituted by halo, cyano, hydroxy, C₁-C₁₀-alkyl, C₁-C₁₀-alkoxy or halo-C₁-C₁₀-alkyl;
C₁-C₁₀-cycloalkyl optionally fused to one or more benzene rings and optionally substituted by C₂-C₁₀-alkyl, C₁-C₁₀-alkoxy, C₃-C₁₀-cycloalkyl, C₇-C₁₄-aralkyl, C₇-C₁₄-aralkyloxy or C₆-C₁₀-aryl, where C₁-C₁₀-cycloalkyl, C₇-C₁₄-aralkyl, C₇-C₁₄-aralkyloxy or C₆-C₁₀-aryl are optionally substituted by halo, hydroxy, C₁-C₁₀-alkyl, C₁-C₁₀-alkoxy or halo-C₁-C₁₀-alkyl;
C₆-C₁₀-aryl optionally substituted by halo, hydroxy, C₁-C₁₀-alkyl, C₁-C₁₀-alkoxy, C₁-C₁₀-haloalkyl, phenoxy, C₁-C₁₀-alkylthio, C₆-C₁₀-aryl, 4- to 10-membered heterocyclic ring having at least one ring nitrogen, oxygen or sulphur atom, or by NR¹⁶R¹⁷ where R¹⁶ and R¹⁷ are each independently C₁-C₁₀-alkyl optionally substituted by hydroxy, C₁-C₁₀-alkoxy or phenyl or R¹⁶ may additionally be hydrogen;
phenoxy optionally substituted by C₁-C₁₀-alkyl, C₁-C₁₀-alkoxy or by phenyl optionally substituted by C₁-C₁₀-alkyl or C₁-C₁₀-alkoxy;
a 4- to 10-membered heterocyclic ring having at least one ring nitrogen, oxygen or sulphur atom, said heterocyclic ring being optionally substituted by halo, C₁-C₁₀-alkyl, C₁-C₁₀-alkoxy, halo-C₁-C₁₀-alkyl, C₆-C₁₀-aryl, C₇-C₁₄-aralkyl, C₇-C₁₄-aralkyloxy, C₁-C₁₀-alkoxycarbonyl or a 4- to 10-membered heterocyclyl-C₁-C₁₀-alkyl;
—NR¹⁸R¹⁹ where R¹⁸ is hydrogen or C₁-C₁₀-alkyl and R¹⁹ is C₁-C₁₀-alkyl optionally substituted by hydroxy, or R¹⁹ is C₈-C₁₀-aryl optionally substituted by halo, or R¹⁹ is a 4- to 10-membered heterocyclic ring having at least one ring nitrogen, oxygen or sulphur atom which ring is optionally substituted by phenyl or halo-substituted phenyl or R¹⁹ is C₆-C₁₀-arylsulfonyl optionally substituted by C₁-C₁₀-alkylamino or di(C₁-C₁₀alkyl)amino;
—SR²⁰ where R²⁰ is C₆-C₁₀-aryl or C₇-C₁₄-aralkyl optionally substituted by halo, C₁-C₁₀-alkyl, C₁-C₁₀-alkoxy or C₁-C₁₀-haloalkyl; or
—CONHR²¹ where R²¹ is C₁-C₁₀-alkyl, C₃-C₁₀-cycloalkyl or C₆-C₁₀-aryl;
   for simultaneous, sequential or separate administration in the treatment of an inflammatory or obstructive airways disease.

In another aspect, the present invention provides a pharmaceutical composition comprising a mixture of effective amounts of (A) as hereinbefore defined and (B) as hereinbefore defined, optionally together with at least one pharmaceutically acceptable carrier.

In a further aspect, the present invention provides a method of treating an inflammatory or obstructive airways disease which comprises administering to a subject in need of such treatment effective amounts of (A) as hereinbefore defined and (B) as hereinbefore defined.

The invention further provides the use of (A) as hereinbefore defined and (B) as hereinbefore defined in the preparation of a medicament for combination therapy by simultaneous, sequential or separate administration of (A) and (B) in the treatment of an inflammatory or obstructive airways disease.

Preferably the molar ratio of (A) to (B) is from 100:1 to 1:300, for example 50:1 to 1:100, especially from 10:1 to 1:20, and more especially from 3:1 to 1:7.

Terms used in the specification have the following meanings:

“Optionally substituted” as used herein means the group referred to can be substituted at one or more positions by any one or any combination of the radicals listed thereafter.

“Halo” or “halogen” is used herein denotes a element belonging to group 17 (formerly group VII) of the Periodic Table of Elements, which may be, for example, fluorine, chlorine, bromine, or iodine. Preferably halo or halogen is fluorine or chlorine.

“C₁-C₁₀-alkyl” as used herein denotes straight chair or branched alkyl that contains one to ten carbon atoms. Preferably, C₁-C₁₀-alkyl is C₁-C₄alkyl.

“C₁-C₁₀-alkylene” as used herein denotes a straight chain or branched alkylene that contains one to ten carbon atoms. Preferably C₁-C₁₀-alkylene is C₁-C₄ alkylene, especially ethylene or methylethylene.

“C₂-C₁₀-alkenyl” as used herein denotes straight chain or branched hydrocarbon chains that contain two to ten carbon atoms and one or more carbon-carbon double bonds. Preferably “C₂-C₁₀-alkenyl” is “C₂-C₄-alkenyl”.

“C₁-C₁₀-alkynyl” at used herein denotes straight chain or branched hydrocarbon chains that contain two to ten carbon atoms and one or more carbon-carbon triple bonds. Preferably “C₂-C₁₀-alkynyl” is “C₂-C₄-alkynyl”.

“5-, 6 or 7-membered carbocyclic ring” as used herein denotes a carbocyclic group having 5to 7 ring carbon atoms, either cycloaliphatic, such as a C₅-C₇-cycloalkyl, or aromatic, such as phenyl, which can be substituted by one or more, usually one or two, C₁-C₄-alkyl groups.

“C₃-C₁₀-cycloalkyl” as used herein denotes cycloalkyl having 3 to 10 ring carbon atoms, for example a monocyclic group such as a cyclopropyl, cyclobutyl, cyclopentyl, cyclohexyl, cycloheptyl, cyclooctyl, cyclononyl or cyclodecyl, any of which can be substituted by one or more, usually one or two, C₁-C₄-alkyl groups, or a bicyclic group such as bicycloheptyl or bicyclooctyl. Preferably C₃-C₁₀-cycloalkyl is C₃-C₆-cycloalkyl, for example cyclopropyl, cyclobutyl, cyclopentyl, cyclohexyl or cycloheptyl.

“C₁-C₁₀-haloalkyl” as used herein denotes C₁-C₁₀-alkyl as hereinbefore defined substituted by one or more halogen atoms, preferably one, two or three halogen atoms.

“C₁-C₁₀-alkylamino” and “di(C₁-C₁₀-alkyl)amino” as used herein denote amino substituted respectively by one or two C₁-C₁₀-alkyl groups as hereinbefore defined, which may be the same or different. Preferably C₁-C₁₀-alkylamino and di(C₁-C₁₀-alkyl)amino are respectively C₁-C₄-alkylamino and di(C₁-C₄-alkyl)amino.

“C₁-C₁₀-alkylthio” as used herein denotes straight chain or branched alkylthio having 1 to 10 carbon atoms. Preferably, C₁-C₁₀-alkylthio is C₁-C₄-alkylthio.

“C₁-C₁₀-alkylthio” as used herein denotes straight chain or branched alkoxy that contains 1 to 10 carbon atoms. Preferably, C₁-C₁₀-alkoxy is C₁-C₄-alkoxy.

“C₁-C₁₀-alkoxy-C₁-C₁₀-alkyl” as used herein denotes C₁-C₁₀-alkyl as hereinbefore defined substituted by C₁-C₁₀-alkoxy. Preferably, C₁-C₁₀-alkoxy-C₁-C₁₀-alkyl is C₁-C₄-alkoxy-C₁-C₄-alkyl.

“C₁-C₁₀-alkoxycarbonyl” as used herein denotes C₁-C₁₀-alkoxy as hereinbefore defined linked through an oxygen atom thereof to a carbonyl group.

“C₆C₁₀-aryl” as used herein denotes a monovalent carbocyclic aromatic group that contains 6 to 10 carbon atoms and which may be, for example, a monocyclic group such as phenyl or a bicyclic group such as naphthyl. Preferably C₆-C₁₀-aryl is C₆-C₈-aryl, especially phenyl.

“C₆-C₁₀-arylsulfonyl” as used herein denotes C₆C₁₀-aryl as hereinbefore defined linked through a carbon atom thereof to a sulfonyl group. Preferably C₆-C₁₀-arylsulfonyl is C₆-C₈-arylsulfonyl.

“C₇-C₂₄-aralkyl” as used herein denotes alkyl, for example C₁-C₄-alkyl as hereinbefore defined, substituted by aryl, for example C₆-C₁₀-aryl as hereinbefore defined. Preferably, C₇-C₁₄-aralkyl is C₇-C₁₀-aralkyl such as phenyl-C₁-C₄-alkyl, particularly benzyl or 2-phenylethyl.

“C₇-C₁₄-aralkyloxy” as used herein denotes alkoxy, for example C₁-C₄-alkoxy as hereinbefore defined, substituted by aryl, for example C₆-C₁₀-aryl. Preferably, C₇-C₁₄-aralkyloxy is C₇-C₁₀-aralkyloxy such as phenyl-C₁-C₄-alkoxy, particularly benzyloxy or 2-phenylethoxy.

Ar as used herein may be, for example, phenylene which is unsubstituted or substituted by one or more substituents selected from halogen, hydroxy, C₁-C₁₀-alkyl, C₁-C₁₀-alkoxy, C₁-C₁₀-alkoxy-C₁-C₁₀-alkyl, phenyl, or C₁-C₁₀-alkyl substituted by phenyl, C₁-C₁₀-alkoxy substituted by phenyl, C₁-C₁₀-alkyl-substituted phenyl and C₁-C₁₀-alkoxy-substituted phenyl. Preferably Ar is phenylene which is unsubstituted or substituted by one or two substituents selected from halogen, C₁-C₄-alkyl, C₁-C₄-alkoxy, or C₁-C₄-alkoxy substituted by phenyl. Preferably one substituent in Ar is para to R¹ and optional second and third substituents in Ar are meta to R¹.

“4- to 10-membered heterocyclic ring having at least one ring nitrogen, oxygen or sulphur atom” as used herein may be, for example, pyrrole, pyrrolidine, pyrazole, imidazole, triazole, tetrazole, thiadiazole, oxazole, isoxazole, thiophene, thiazole, isothiazole, oxadiazole, pyridine, pyrazine, pyridazine, pyrimidine, piperidine, piperazine, triazine, oxazine, morpholino, quinoline, isoquinoline, naphthyridine, indane or indene. Preferred heterocyclic rings include thiazole, pyrrolidine, piperidine, azacycloheptane and isoxazole.

“4 to 10-membered heterocylyl-C₁-C₁₀-alkyl” denotes alkyl, for example C₁-C₁₀-alkyl as hereinbefore defined, substituted by a 4- to 10-membered heterocyclic ring as hereinbefore defined. Preferably, 4- to 10-membered heterocyclyl-C₁-C₁₀-alkyl is C₁-C₄-alkyl substituted by a 4- to 8-membered heterocyclic ring having at least one ring nitrogen, oxygen or sulphur atom.

“C₁-C₄-alkylsulfonyl” denotes sulfonyl substituted by C₁-C₄-alkyl as hereinbefore defined. “Hydroxy-C₁-C₄-alkyl” denotes C₁-C₄-alkyl as hereinbefore defined substituted by one or more, preferably one, two or three hydroxy groups.

R¹³ and R¹⁴ together with the carbon atoms to which they are attached as a cycloaliphatic ring may be, for example, a cyclopentane ring, optionally substituted by one or two C₁-C₄-alkyl groups, a cyclohexane ring, optionally substituted by one or two C₁-C₄-alkyl groups, or a cycloheptane ring, preferably a cyclopentane ring.

In one aspect, the present invention provides a medicament, comprising, separately or together (A) glycopyrrolate; and (B) either a compound of formula I as hereinbefore defined or a compound of formula II as hereinbefore defined; for simultaneous, sequential or separate administration in the treatment of an inflammatory or obstructive airways disease.

(A) Glycopyrrolate is a known antimuscarinic agent. More specifically it inhibits acetyl choline binding to M3 muscarinic receptors thereby inhibiting bronchoconstriction.

Glycopyrrolate is a quaternary ammonium salt. Suitable counter ions are pharmaceutically acceptable counter ions including, for example, fluoride, chloride, bromide, iodide, nitrate, sulfate, phosphate, formate, acetate, trifluoroacetate, propionate, butyrate, lactate, citrate, tartrate, malate, maleate, succinate, benzoate, p-chlorobenzoate, diphenyl-acetate or triphenylacetate, o-hydroxybenz-oate, p-hydroxybenzoate, 1-hydroxynaphthalene-2-carboxylate, 3-hydroxynaphthalene-2-carboxylate, methanesulfonate and benzenesulfonate. Its bromide salt, namely 3-[(cyclopentyl-hydroxyphenylacetyl)oxy]-1,1-dimethylpyrrolidinium bromide, has the following structural formula

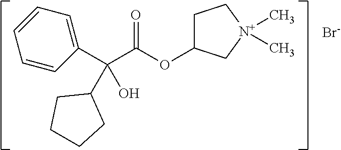

and can be prepared using the procedures described in U.S. Pat. No. 2,956,062.

Glycopyrrolate has two stereogenic centres and hence exists in four isomeric forms, namely (3R,2′R)-, (3S,2′R)-, (3R,2′S)- and (3S,2′S)-3-[(cyclopentyl-hydroxyphenylacetyl)oxy]-1,1-dimethylpyrrolidinium bromide, as described in United States patent specifications U.S. Pat. Nos. 6,307,060 and 6,613,795. The contents of these patent specifications is incorporated herein by reference. The present invention embraces using one or more of these isomeric forms, especially the 3S,2′R isomer, the 3R,2′R isomer or the 2S,3′R isomer, thus including single enantiomers, or racemates, especially the (3S,2′R/2S,3′R) racemate.

(B) is either a compound of formula I as hereinbefore defined or a compound of formula II as hereinbefore defined. Compounds of these formulas possess beta-2 adrenoceptor agonist activity. They commonly have a rapid onset of action and have a prolonged stimulating action on the β₂-adrenoceptor, for example up 24 hours or longer.

Preferred compounds of formula I include those wherein
R⁸, R⁹ and R¹⁰ are each H, R¹ is OH, R² and R³ are each H and
(i) R^{x} and R^{y} are both —CH₁—, and R⁴ and R⁷ are each CH₃O— and R⁵ and R⁶ are each H;
(ii) R^{x} and R^{y} are both —CH₂—, and R⁴ and R⁷ are each H and R⁵ and R⁶ are each CH₃CH₂—;
(iii) R^{x} and R^{y} are both —CH₂—, and R⁴ and R⁷ are each H and R⁵ and R⁶ are each CH₃—;
(iv) R^{x} and R^{y} are both —CH₂—, and R⁴ and R⁷ are each CH₃CH₂— and R⁵ and R⁶ are each H;
(v) R^{x} and R^{y} are both —CH₂—, and R⁴ and R⁷ are each H and R⁵ and R⁶ together denote —(CH₂)₄—;
(vi) R^{x} and R^{y} are both —CH₂—, and R⁴ and R⁷ are each H and R⁵ and R⁶ together denote —O(CH₂)₂O—;
(vii) R^{x} and R^{y} are both —CH₂—, and R⁴ and R⁷ are each H and R⁵ and R⁶ are each CH₃(CH₂)₃—;
(viii) R^{x} and R^{y} are both —CH₂—, and R⁴ and R⁷ are each H and R⁵ and R⁶ are each CH₃(CH₂)₂—;
(ix) R^{x} and R^{y} are both —(CH₂)₂—, R⁴, R⁵, R⁶ and R⁷ are each H; or
(x) R^{x} and R^{y} are both —CH₂—, and R⁴ and R⁷ are each H and R⁵ and R⁶ are each CH₃OCH₂—; or

Especially preferred compounds of formula I include 8-hydroxy-S-[1-hydroxy-2-(indan-2-yl-amino)-ethyl]-1H-quinolin-2-one, 5-[2-(5,6-dimethoxy-indan-2-ylamino)-1-hydroxy-ethyl]-8-hydroxy-1H-quinolin-2-one, 5-[2-(5,6-diethyl-indan-2-ylamino)-1-hydroxy-ethyl]-8-hydroxy-3-methyl-1H-quinolin-2-one, 5-[2-(5,6-diethyl-indan-2-ylamino)-1-hydroxy-ethyl]-8-methoxy-methoxy-6-methyl-1H-quinolin-2-one, 5-[2-(5,6-diethyl-indan-2-ylamino)-1-hydroxy-ethyl]-8-hydroxy-6-methyl-1H-quinolin-2-one, 8-hydroxy-S-[2-(5,6diethyl-indan-2-ylamino)-1-hydroxy-ethyl]-3,4-dihydro-1H-quinolin-2-one, 5-[(R)-2-(5,6-diethyl-2-methyl-indan-2-yl-amino)-1-hydroxy-ethyl]-8-hydroxy-1H -quinolin-2-one, (S)-S-[2-(4,7-diethyl-indan-2-ylamino)-1-hydroxy-ethyl]-8-hydroxy-1H-quinolin-2-one hydrochloride, 5-[(R)-1-hydroxy-2-(6,7,8,9-tetrahydro-5H-benzocyclohepten-7-ylamino)-ethyl]-8-hydroxy-1H-quinolin-2-one hydrochloride, (R)-5-[2-(5,6-diethyl-indan-2-ylamino)-1-hydroxy-ethyl]-8-hydroxy-1H-quinolin-2-one maleate, (R)-S-[2-(5,6diethyl-indan-2-ylamino)-1-hydroxy-ethyl]-8-hydroxy-1H-quinolin-2-one hydrochloride, (R)-8-hydroxy-5-[(S)-1-hydroxy-2-(4,5,6,7-tetramethyl-indan-2-ylamino)-ethyl]-1H-quinolin-2-one, 8-hydroxy-5-[(R)-1-hydroxy-2-(2-methyl-indan-2-ylamino)-ethyl]-1H-quinolin-2-one, 5-[2-(5,6-diethyl-indan-2-ylamino)-ethyl]-8-hydroxy-1H-quinolin-2-one, 8-hydroxy-5-[(R)-1-hydroxy-2-(2-methyl-2,3,5,6,7,8-hexahydro-1H-cyclopenta[b]naphthalen-2-ylamino)-ethyl]-1H-quinolin-2-one, and 5-[(S)-2-(2,3,5,6,7,8-hexahydro-1H-cyclopenta[b]naph-thalen-2-ylamino)-1-hydroxy-ethyl]-8-hydroxy-1H-quinolin-2-one,

Compounds of formula I in free or salt or solvate form may be prepared by using the procedures described in international patent applications WO 2000/075114, WO 2003/076387, WO 2004/076422 or WO 2004/087668, the contents of which are incorporated herein by reference.

Compounds of formula I in free form may be converted into salt form, and vice versa, in a conventional manner. The compounds in free or salt form can be obtained in the form of hydrates or solvates containing a solvent used for crystallisation. Compounds of formula I can be recovered from reaction mixtures and purified in a conventional manner. Isomers, such as enantiomers, may be obtained in a conventional manner, e.g. by fractional crystallisation or asymmetric synthesis from correspondingly asymmetrically substituted, e.g. optically active, starting materials.

Preferred compounds of formula II include those wherein
X is —R¹³—Ar—R¹⁴ or —R¹⁵—Y;
Ar denotes a phenylene group optionally substituted by halo, C₁-C₁₀-alkyl, C₁-C₁₀-alkoxy or by C₁-C₁₀-alkoxy substituted by phenyl;
R¹³ and R¹⁴ are attached to adjacent carbon atoms in Ar, and
either R¹³ to C₁-C₁₀-alkylene and R¹⁴ to hydrogen,
or R¹³ and R¹⁴ together with the carbon atoms in Ar to which they are attached denote a 5-, 6- or 7-membered cycloaliphatic ring;
R¹⁵ is a bond or C₁-C₁₀-alkylene optionally substituted by hydroxy, C₆-C₁₀-aryl or C₇-C₁₄-aralkyl; and
Y is C₁-C₁₀-alkyl, C₁-C₁₀-alkoxy or C₁-C₁₀-alkynyl; C₁-C₁₀-cycloalkyl optionally fused to one or more benzene rings and optionally substituted by C₁-C₁₀-alkyl, C₃-C₁₀-cycloalkyl, C₇-C₁₄-aralkyl, C₇-C₁₄-aralkyloxy optionally substituted by halo, or by C₆-C₁₀-aryl optionally substituted by C₁-C₁₀-alkyl or C₁-C₁₀-alkoxy; C₆-C₁₀-aryl optionally substituted by halo, hydroxy, C₁-C₁₀-alkyl, phenoxy, C₁-C₁₀-alkylthio, C₁-C₁₀-aryl, a 4- to 10-membered heterocyclic ring having at least one ring nitrogen atom, or by NR¹⁶R¹⁷ where R¹⁶ and R¹⁷ are each independently C₁-C₁₀-alkyl optionally substituted by hydroxy or phenyl or R¹⁶ may additionally be hydrogen; phenoxy optionally substituted by C₁-C₁₀-alkoxy; a 4- to 10-membered heterocyclic ring having at least one ring nitrogen or oxygen atom, said heterocyclic ring being optionally substituted by C₁-C₁₀-alkyl, C₆-C₁₀-aryl, C₇-C₁4-aralkyl, C₁-C₁₀-alkoxycarbonyl or by a 4- to 10-membered heterocyclyl-C₁-C₁₀-alkyl; —NR¹⁸R¹⁹ where R¹⁸ is hydrogen or C₁-C₁₀-alkyl and R¹⁹ is C₁-C₁₀-alkyl, or R¹⁹ is a 4-to 10-membered heterocyclic ring having at least one ring nitrogen or oxygen atom which ring is optionally substituted by halo-substituted phenyl or R¹⁹ is C₆-C₁₀-arylsulfonyl optionally substituted by di(C₁-C₁₀-alkyl)amino; —SR²⁰ where R²⁰ is C₆-C₁₀-aryl or C₇-C₁₄-aralkyl optionally substituted by halo or C₁-C₁₀-haloalkyl; or —CONHR²¹ where R²² is C₈-C₁₀-cycloalkyl or C₆-C₁₀-aryl.

Especially preferred compounds of formula II include those wherein
X to —R¹³—Ar—R¹⁴ or —R¹⁵—Y;
Ar denotes a phenylene group optionally substituted by halo, C₁-C₆-alkyl, C₁-C₄-alkoxy or by C₁-C₄-alkoxy substituted by phenyl;
R¹³ and R¹⁴ are attached to adjacent carbon atoms in Ar, and
either R¹³ to C₁-C₄-alkylene and R¹⁴ to hydrogen,
or R¹³ and R¹⁴ together with the carbon atoms in Ar to which they are attached denote a 5-, 6- or 7-membered cycloaliphatic ring, especially a 5-membered cycloaliphatic ring;
R¹⁵ is a bond or C₁-C₄-alkylene optionally substituted by hydroxy, C₆-C₈-aryl or C₇-C₁₀-aralkyl; and
Y is C₁-C₄-alkyl, C₁-C₄-alkoxy or C₁-C₄-alkynyl; C₃-C₆-cycloalkyl optionally fused to one or more benzene rings and optionally substituted by C₁-C₁₀-alkyl, C₃-C₆-cycloalkyl, C₇-C₂₀-aralkyl, C₇-C₁₀-aralkyloxy optionally substituted by halo, or by C₆-C₈-aryl optionally substituted by C₁-C₄-alkyl or C₁-C₄-alkoxy; C₆-C₈-aryl optionally substituted by halo, hydroxy, C₁-C₄-alkyl, phenoxy, C₁-C₄-alkylthio, C₆-C₈-aryl, a 4- to 8-membered heterocyclic ring having at least one ring nitrogen atom, or by NR¹⁶R¹⁷ where R¹⁶ and R¹⁷ are each independently C₁-C₄-alkyl optionally substituted by hydroxy or phenyl or R¹⁶ may additionally be hydrogen; phenoxy optionally substituted by C₁-C₄-alkoxy; a 4- to 8-membered heterocyclic ring having at least one ring nitrogen or oxygen atom, said heterocyclic ring being optionally substituted by C₁-C₄-alkyl, C₆-C₈-aryl, C₇-C₁₀-aralkyl, C₁-C₄-alkoxycarbonyl or by a 4- to 8-membered heterocyclyl-C₁-C₄-alkyl; —NR¹⁸R¹⁹ where R¹⁸ is hydrogen or C₁-C₄-alkyl and R¹⁹ is C₁-C₄-alkyl, or R¹⁹ is a 4- to 8-membered heterocyclic ring having at least one ring nitrogen or sulphur atom which ring is optionally substituted by halo-substituted phenyl or R¹⁹ is C₆-C₈-arylsulfonyl optionally substituted by di(C₁-C₄-alkyl)amino; —SR²⁰ where R²⁰ is C₆-C₈-aryl or C₇-C₁₀-aralkyl optionally substituted by halo or C₁-C₄-haloalkyl; or —CONHR²¹ where R²² is C₃-C₆-cycloalkyl or C₆-C₈-aryl.

More especially preferred compounds of formula II include 4-hydroxy-7-(1-hydroxy-2-(2-[4-(4-phenyl-butoxy)-phenyl]-ethylamino)-ethyl)-3H-benzothiazol-2-one; 7-[(R)-2-(1,1-Dimethyl-2-phenyl-ethylamino)-1-hydroxy-ethyl]-4-hydroxy-3H-benzothiazol-2-one; 4-Hydroxy-7-((R)-1-hydroxy-2-[2-(5,6,7,8-tetrahydro-naphthalen-2-yl)-ethylamino]-ethyl]-3H-benzothiazol-2-one formate; 7-[(R)-2-((1S,2S)-2-Benzyloxy-cyclopentylamino)-1-hydroxy-ethyl]-4-hydroxy-3H-benzo-thiazol-2-one; and 7-[(R)-2-((1S,2R)-2-Benzyloxy-cyclopentylamino)-1-hydroxy-ethyl]-4-hydroxy-3H-benzothiazol-2-one.

In formula II the carbon atom alpha to the phenolic ring carries a hydroxy group and so is asymmetric, so the compound exists in individual optically active isomeric forms or as mixtures thereof, e.g. as racemic or diastereomeric mixtures. Compounds of formula II embrace both individual optically active R and S isomers as well as mixtures, e.g. racemic or diastereomeric mixtures, thereof.

Compounds of formula II in free or salt or solvate form may be prepared by using the procedures described in international patent application WO 2004/016601, the contents of which, is incorporated herein by reference.

Pharmaceutically acceptable acid addition salts of the compounds of formulae I and II include those of inorganic acids, for example, hydrohalic acids such as hydrofluoric acid, hydrochloric acid, hydrobromic acid or hydroiodic acid, nitric acid, sulfuric acid, phosphoric acid; and organic acids, for example aliphatic monocarboxylic acids such as formic acid, acetic acid, trifluoro-acetic acid, propionic acid and butyric acid, aliphatic hydroxy acids such as lactic acid, citric acid, tartaric acid or malic acid, dicarboxylic acids such as maleic acid or succinic acid, aromatic carboxylic acids such as benzoic acid, p-chlorobenzoic acid, dipbenylacetic acid or triphenylacetic acid, aromatic hydroxy acids such as o-hydroxybenzole acid, p-hydroxy-benzoic acid, 1-hydroxynaphthalene-2-carboxylic acid or 3-hydroxynaphthalene-2-carboxylic acid, and sulfonic acids such as methanesulfonic acid or benzenesulfonic acid. These salts may be prepared by known salt-forming procedures. Pharmaceutically acceptable solvates are generally hydrates. Isomers, such as enantiomers, may be obtained in a conventional manner, e.g. by fractional crystallisation or asymmetric synthesis from correspondingly asymmetrically substituted, e.g. optically active, starting materials.

The medicament of the present invention may additionally contain one or more co-therapeutic agents such as anti-inflammatory, bronchodilatory, antihistamine, decongestant or anti-tussive drug substances, particularly in the treatment of obstructive or inflammatory airways diseases such as those mentioned hereinbefore, for example as potentiators of therapeutic activity of such drugs or as a means of reducing required dosaging or potential side effects of such drugs.

Co-therapeutic agents include steroids, A_{2A} agonists, A_{2A} antagonists, antihistamines, caspase inhibitors, LTB4 antagonists, LTD4 antagonists, PDE4 inhibitors, mucolytics, matrix metal loproteinase inhibitors (MMPi's), leukotrienes, antibiotics, anti neoplastics, peptides, vaccines, nicotine, elastase inhibitors and sodium cromoglycate.

Such anti-inflammatory drugs include steroids, for example glucocorticosteroids such as budesonide, heclamethasone dipropionate, fluticasone propionate, ciclesonide or mometasone furoate, or steroids described in WO 02/88167, WO 02/12266, WO 02/100379, WO 02/00679 (especially those of Examples 3, 11, 14, 17, 19, 26, 34, 37, 39, 51, 60, 67, 72, 73, 90, 99 and 101), WO 03/35668, WO 03/48181, WO 03/62259, WO 03/64445, WO 03/72592, WO 04/39827 and WO 04/66920, and non-steroidal glucocorticoid receptor agonists, such as those described in DE 10261874, WO 00/00531, WO 02/10143, WO 03/82280, WO 03/82787, WO 03/86294, WO 03/104195, WO 03/101932, WO 04/05229, WO 04/18429, WO 04/19935, WO 04/26248, WO 0505452. Suitable A_{2A} agonists include those described in EP 409595A2, EP 1052264, EP 1241176, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462, WO 03/086408, WO 04/039762, WO 04/039766, WO 04/045618 and WO 04/046083. Suitable A_{2B} antagonists include those described in WO 03/042214 and WO 02/42298. Suitable antihistamine drug substances include cetirizine hydrochloride, acetaminophen, elemastine fumarate, promethazine, loratidine, desloratidine, diphenhydramine and fexofenadine hydrochloride, activastine, asternizole, azelastine, ebastine, epinastine, mizolastine and teferiadine as well as those disclosed in WO 03/099807, WO 04/026841, JP 2004107299. Suitable caspase inhibitors, including interleukin-IP converting enzyme (ICE) inhibitors, include those that are disclosed in Canadian patent specification 2109646, EP 519748, EP 547 699, EP 590 650, EP 628550, EP 644 197, EP 644198, WO 93/05071, WO 93/14777, WO 93/16710, WO 94/00154, WO 94/03480, WO 94/21673, WO 95/05152, WO 95/35308, WO 97/22618, WO 97/22619, WO 98/41232, WO 99/06367, WO 99/65451, WO 01/119373, U.S. Pat. No. 5,411,985, U.S. Pat. Nos. 5,416,013, 5,430,128, 5,434,248, 5,565,430, 5,585,357, 5,656,627, 5,677,283, 6,054,487, 6,531,474, US 20030096737, GB 2,278,276 as well as those disclosed in international parent applications WO 98/10778, WO 98/11109, WO 98/11129 and WO 03/32918. Suitable LTB4 antagonists include LY293111, CGS025019C, CP-195543, SC-53228, BIIL 284, ONO 4057, SB 209247 and those described in U.S. Pat. No. 5,451,700 and WO 04/108720. Suitable LTD4 antagonists include montalukast and zafirlokast. Suitable PDE4 inhibitors PDE4 inhibitors such as cilomilast (Ariflo® GlaxoSmithKline), Roflumilast (Byk Gulden), V-11294A (Napp), BAY19-8004 (Bayer), SCH-351591 (Schering-Plough), Arofylline (Almirali Prodesfarma), PD189659/PD168787 (Parke-Davis), AWD-12-281 (Asta Medica), CDC-801 (Celgene), SelCID(TM) CC-10004 (Celgene), VM554/UM565 (Vernalis), T-440 (Tanabe), KW-4490 (Kyowa Hakko Kogyo), GRC 3886 (Glenmark), and those described in WO 92/19594, WO 93/19749, WO 93/19750, WO 93/19751, WO 98/18796, WO 99/16766, WO 01/13953, WO 03/104204, WO 03/104205, WO 03/39544, WO 04/000814, WO 04/000839 and WO 04/005258, WO 04018450, WO 04/018451,WO 04/018457, WO 04/018465, WO 04/018431, WO 04/018449, WO 04/018450, WO 04/013451, WO 04/018457, WO 04/018465, WO 04/019944, WO 04/019945, WO 04/045607, WO 04/037805, WO 04/063197, WO 04/103998 and WO 04/111044.

While (A) glycopyrrolate is an M3 antagonist, the medicament of the present invention optionally includes one or more other M3 antagonists such as ipratropium bromide, oxitropium bromide, tiotropium salt, CHF 4226 (Chiesi), or those described in WO 02/51841, WO 02/53564, WO 03/00840, WO 03/87094, WO 04/05285, WO 02/00652, WO 03/53966, EP 424021, U.S. Pat. Nos. 5,171,744, 3,714,357, WO 03/33495, WO 04/018422 or WO 05/003090.

While (B) are beta-2 adrenoceptor agonists, the medicament of the present invention optionally includes one or more other beta-2 adrenoceptor agonists such as albuterol (salbutamol), metaproterenol, terbutaline, salmeterol fenoterol, procaterol, and especially, formoterol, carmoterol and pharmaceutically acceptable salts thereof, compounds (in free or sale or solvate form) of formula I of WO 04/087142, or those described in JP 0502504S, US 2002/0055651, WO 93/18007, WO 99/64035, WO 01/42193, WO 01/83462, WO 02/066422, WO 02/070490, WO 02/076933, WO 03/24439, WO 03/72539, WO 03/42160, WO 03/91204, WO 03/42164, WO 03/99764, WO 04/11416, WO 04/16578, WO 04/22547, WO 04/32921, WO 04/33412, WO 04/37773, WO 04/37807, WO 04/39762, WO 04/39766, WO 04/45618, WO 04/46083, WO 04/80964, WO 04/108675 or WO 04/108676.

Administration of the medicament or pharmaceutical composition as hereinbefore described, i.e. with (A) and (B) in admixture or separate, is preferably by inhalation, i.e. (A) and (B) are in inhalable form. The inhalable form of the medicament may be, for example, an atomizable composition such as an aerosol comprising the active ingredient, i.e. (A) and (B) separately or in admixture, in solution or dispersion in a propellant, or a nebulizable composition comprising a solution or dispersion of the active ingredient in an aqueous, organic or aqueous/organic medium. For example, the inhalable form of the medicament may be an aerosol comprising a mixture of (A) and (B) in solution or dispersion in a propellant, or a combination of an aerosol containing (A) in solution or dispersion in a propellant with an aerosol containing (B) in solution or dispersion in a propellant. In another example, the inhalable form is a nebulizable composition comprising a dispersion of (A) and (B) in an aqueous, organic or aqueous/organic medium, or a combination of a dispersion of (A) in such a medium with a dispersion of (B) in such a medium.

An aerosol composition suitable for use as the inhalable form of the medicament may comprise the active ingredient in solution or dispersion in a propellant, which may be chosen from any of the propellants known in the art. Suitable such propellants include hydrocarbons such as n-propane, n-butane or isobutane or mixtures of two or more such hydrocarbons, and halogen-substituted hydrocarbons, for example chlorine and/or fluorine-substituted methanes, ethanes, propanes, butanes, cyclopropanes or cyclobutanes, such as dichlorodifluoromethane (CPC 12), trichlorofluoromethane (CFC11), 1,2-dichloro-1,1,2,2-tetrafluoroethane (CFC114) or, particularly, 1,1;1,2-tetrafluoroerhane (HFA134a) and 1,1,1,2,3,3,3-heptafluoropropane (HFA227), or mixtures of two or more such halogen-substituted hydrocarbons. Where the active ingredient is present in suspension in the propellant, i.e. where it is present in particulate form dispersed in the propellant, the aerosol composition may also contain a lubricant and a surfactant, which may be chosen from those lubricants and surfactants known in the art. Other suitable aerosol compositions include surfactant-free or substantially surfactant-free aerosol compositions. The aerosol composition may contain up to about 5% by weight, for example 0.0001 to 5%, 0.001 to 5%, 0.001 to 3%, 0.001 to 2%, 0.001 to 1%, 0.001 to 0.1%, or 0.001 to 0.01% by weight of the active ingredient, based on the weight of the propellant. Where present, the lubricant and surfactant may be in an amount up to 5% and 0.5% respectively by weight of the aerosol composition. The aerosol composition may also contain a co-solvent such as ethanol in an amount up to 30% by weight of the composition, particularly for administration from a pressurised metered dose inhalation device. The aerosol composition may further contain a bulking agent, for example a sugar such as lactose, sucrose, dextrose, mannitol or sorbitol, in an amount, for example, of up to 20%, usually 0.001 to 1%, by weight of the composition.

In another embodiment of the invention, the inhalable form is a dry powder, i.e. (A) and (B) are present in a dry powder comprising finely divided (A) and (B) optionally together with at least one particulate pharmaceutically acceptable carrier, which may be one or more materials known as pharmaceutically acceptable carriers, preferably chosen from materials known as carriers in dry powder inhalation compositions, for example saccharides, including monosaccharides, disaccharides, polysaccharides and sugar alcohols such as arabinose, glucose, fructose, ribose, mannose, sucrose, trehalose, lactose, maltose, starches, dextran, mannitol or sorbitol. An especially preferred carrier is lactose. The dry powder may be contained as unit doses in capsules of, for example, gelatin or plastic, or in blisters (e.g. of aluminium or plastic), for use in a dry powder inhalation device, which may be a single dose or multiple dose device, preferably in dosage units of (A) and (B) together with the carrier in amounts to bring the total weight of powder per capsule to from 5 mg to 50 mg. Alternatively, the dry powder may be contained in a reservoir in a multi-dose dry powder inhalation device adapted to deliver, for example, 3-25 mg of dry powder per actuation.

In the finely divided particulate form of the medicament, and in the aerosol composition where the active ingredient is present in particulate form, the active ingredient may have an average particle diameter of up to about 10 μm, for example 0-1 to 5 μm, preferably 1 to 5 μm. The particulate carrier, where present, generally has a maximum particle diameter up to 300 μm, preferably up to 212 μm, and conveniently has a mean particle diameter of 40 to 100 μm, e.g. 50 to 75 μm. The particle size of the active ingredient, and that of a particulate carrier where present in dry powder compositions, can be reduced to the desired level by conventional methods, for example by grinding in an air-jet mill, ball mill or vibrator mill, sieving, microprecipitation, spray-drying, lyophilisation or controlled crystallisation from conventional solvents or from supercritical media.

The medicament may be a controlled release formulation comprising finely divided particles of (A) and (B) within a hydrophobic matrix material, e.g. comprising magnesium stearate, for example as described in international patent application WO 01/76575, the contents of which is incorporated herein by reference.

The inhalable medicament may be administered using an inhalation device suitable for the inhalable form, such devices being well known in the art. Accordingly, the invention also provides a pharmaceutical product comprising a medicament or pharmaceutical composition, as hereinbefore described in inhalable form as hereinbefore described in association with one or more inhalation devices. In a further aspect, the invention provides an inhalation device, or a pack of two or more inhalation devices, containing a medicament or pharmaceutical composition as hereinbefore described in inhalable form as hereinbefore described.

Where the inhalable form of the active ingredient is an aerosol composition, the inhalation device may be an aerosol vial provided with a valve adapted to deliver a metered dose, such as 10 to 100 μl, e.g. 25 to 50 μl, of the composition, i.e. a device known as a metered dose inhaler. Suitable such aerosol vials and procedures for containing within them aerosol compositions under pressure are well known to those skilled in the art of inhalation therapy. For example, an aerosol composition may be administered from a coated can, for example as described in EP 0642992 A.

Where the inhalable form of the active ingredient is a nebulizable aqueous, organic or aqueous/organic dispersion, the inhalation device may be a known nebulizer, for example a conventional pneumatic nebulizer such as an airjet nebulizer, or an ultrasonic nebulizer, which may contain, for example, from 1 to 50 ml, commonly 1 to 10 ml, of the dispersion; or a hand-held nebulizer, sometimes referred to as a soft mist or soft spray inhaler, for example an electronically controlled device such as an AERx (Aradigm, US) or Aerodose (Aerogen), or a mechanical device such as a RESPIMAT (Boehringer Ingelheim) nebulizer which allows much smaller nebulized volumes, e.g. 10 to 100 μl, than conventional nebulizers.

Where the inhalable form of the active ingredient is the finely divided particulate form, the inhalation device may be, for example, a dry powder inhalation device adapted to deliver dry powder from a capsule or blister containing a dry powder comprising a dosage unit of (A) and (B) or a multidose dry powder inhalation (MDPI) device adapted to deliver, for example, 3-25 mg of dry powder comprising a dosage unit of (A) and (B) per actuation. The dry powder formulation preferably contains the active ingredients optionally together with a diluent or carrier, such as lactase, of the desired particle size distribution and a compound that helps to protect against product performance deterioration due to moisture e.g. magnesium stearate. Suitable such dry powder inhalation devices are well known. For example, a suitable device for delivery of dry powder in encapsulated form is that described in U.S. Pat. No. 3,991,761, while a suitable MDPI device is that described in WO 97/20589.

The medicament of the invention is preferably a pharmaceutical composition comprising a mixture of (A) as hereinbefore defined and (B) as hereinbefore defined, preferably together with at least one pharmaceutically acceptable carrier as hereinbefore described.

The molar ratio of (A) to (B) may be, in general, from 100:1 to 1:300, for example from 50:1 to 1:100 or from 20:1 to 1:50, preferably from 10:1 to 1:20, more preferably from 5:1 to 1:10, from 3:1 to 1:7 or from 2:1 to 1:2. The compound (A) and the compound (B) may be administered separately in the same ratio.

A suitable daily dose of the compound (A), particularly as the bromide salt, for inhalation may be from 10 μg to 2000 μg, preferably from 60 to 1000 μg, and especially from 80 to 800 μg, e.g. from 20 to 500 μg.

A suitable daily dose of compound (B) for inhalation, may be from 10 μg to 2000 μg, for example from 10 to 1500 μg, from 10 to 1000 μg, preferably from 20 to 800 μg, e.g. from 20 to 600 μg or from 20 to 500 μg.

A suitable unit dose of compound (A), particularly as the bromide salt, may be from 10 μg to 2000 μg, preferably from 60 to 1000 μg, especially from 80 to 800 μg, e.g. from 20 to 500 μg.

A suitable unit dose of compound (B) may be from 10 μg to 2000 μg, for example from 10 to 1500 μg, from 10 to 1000 μg, preferably from 20 to 800 μg, e.g. from 20 to 600 μg or from 20 to 500 μg.

These unit doses may be administered once or twice daily in accordance with the daily doses mentioned hereinbefore. A single dose is preferred. The precise unit and daily dose used will of course depend on the condition to be treated, the patient and the efficiency of the inhalation device.

In one preferred embodiment of the invention, the medicament of the invention is a pharmaceutical composition which is a dry powder in a capsule containing a unit dose of (A) and (B), for example for inhalation from a single capsule inhaler, the capsule suitably containing a unit dose of (A) e.g. as hereinbefore described, and a unit dose of (B), e.g. as hereinbefore described, together with a pharmaceutically acceptable carrier as hereinbefore described in an amount to bring the total weight of dry powder per capsule to between 5 mg and 50 mg, for example 5 mg, 10 mg, 15 mg, 20 mg, 25 mg, 30 mg, 35 mg, 40 mg, 45 mg or 50 mg.

In another preferred embodiment of the invention, the medicament of the invention is a pharmaceutical composition which is a dry powder for administration from a reservoir of a multi-dose dry powder inhaler adapted to deliver, for example, 3 mg to 25 mg of powder containing a unit dose of (A) and (B) per actuation, for example, where (A) is in the form of the maleate salt, a powder comprising, by weight, 20 to 2000 parts, for example 60 to 1000 parts, 100 to 500 parts, or 100 to 300 parts of (A); 25 to 300 parts, e.g. 25 to 500 parts, 50 to 400 parts, or 100 to 400 parts of (B); 0.2 to 1 part magnesium stearate, and 2000 to 25000 parts, e.g. 4000 to 15000 parts or 4000 to 10000 parts of a pharmaceutically acceptable carrier as hereinbefore described.

In a further preferred embodiment of the invention, the medicament of the invention is a pharmaceutical composition which is an aerosol comprising (A) and (B) e.g. in a ratio as hereinbefore described, in a propellant as hereinbefore described, optionally together with a surfactant and/or a bulking agent and/or a co-solvent such as ethanol as hereinbefore described, for administration from a metered dose inhaler adapted to deliver an amount of aerosol containing a unit dose of (A) and a unit dose of (B), or a known fraction of a unit dose of (A) and a known fraction of a unit dose of (B), per actuation. Thus if, for example, the inhaler delivers half of the unit doses of (A) and (B) per actuation, the unit doses can be administered by two actuations of the inhaler.

In accordance with the above, the invention also provides a pharmaceutical kit comprising (A) and (B) as hereinbefore defined in separate unit dosage forms, said forms being suitable for administration of (A) and (B) in effective amounts. Such a kit suitably further comprises one or more inhalation devices for administration of (A) and (B). For example, the kit may comprise one or more dry powder inhalation devices adapted to deliver dry powder from a capsule, together with capsules containing a dry powder comprising a dosage unit of (A) and capsules containing a dry powder comprising a dosage unit of (B). In another example, the kit may comprise a multidose dry powder inhalation device containing in the reservoir thereof a dry powder comprising (A) and a multidose dry powder inhalation device containing in the reservoir thereof a dry powder comprising (B). In a further example, the kit may comprise a metered dose inhaler containing an aerosol comprising (A) in a propellant and a metered dose inhaler containing an aerosol comprising (B) in a propellant.

The medicaments of the invention are advantageous in the treatment of inflammatory or obstructive airways disease, exhibiting highly effective bronchodilatory and anti-inflammatory properties. For instance, it is possible using the combination therapy of the invention to reduce the dosages of (A) or (B) required for a given therapeutic effect compared with those required using treatment with either (A) or (B) alone, thereby minimising possibly undesirable side effects. Furthermore, using the combinations of the invention, particularly using compositions containing (A) and (B), medicaments which have a rapid onset of action and a long duration of action may be prepared. Moreover, using such combination therapy, medicaments which result in a significant improvement in lung function may be prepared. In another aspect, using the combination therapy of the invention, medicaments which provide effective control of obstructive or inflammatory airways diseases, or a reduction in exacerbations of such diseases, may be prepared. In a further aspect, using compositions of the invention containing (A) and (B), medicaments which reduce or eliminate the need for treatment with short-acting rescue medicaments such as salbutamol or terbutaline, may he prepared; thus compositions of the invention containing (A) and (B) facilitate the treatment of an obstructive or inflammatory airways disease with a single medicament.

Treatment of inflammatory or obstructive airways diseases in accordance with the invention may be symptomatic or prophylactic treatment. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitis asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”).

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, for example anti-inflammatory (e.g. corticosteroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e, at a time normally substantially distant form any previously administered symptomatic asthma therapy.

Other inflammatory or obstructive airways diseases and conditions to which the present invention is applicable include acute/adult lung injury (ALI), adult/acute respiratory distress syndrome (ARDS), cystic fibrosis, chronic obstructive pulmonary, airways or lung disease (COPD, COAD or COLD), including chronic bronchitis and emphysema, bronchlectasis and exacerbation of airways hyperreactivity consequent to other drug therapy, in particular other inhaled drug therapy. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tobacosis and byssinosis.

The invention is illustrated by the following Examples.

### EXAMPLES

### Compound A1

3-[(cyclopentyl-hydroxyphenylacetyl)oxy]-1,1-dimethylpyrrolidinium bromide (glycopyrrolate) This compound is commercially available as a racemate or is prepared using the procedures described in U.S. Pat. No. 2,956,062.

### Compound B1

(R)-5-[2-(5,6-diethyl-indan-2-ylamino)-1-hydroxy-ethyl]-8-hydroxy-1H-quinolin-2-one maleate This compound is prepared using the procedures described in international patent application WO 2000/075114.

### Compound B2 to B6

4-hydroxy-7-(1-hydroxy-2-[2-[4-(4-phenyl-butoxy)-phenyl]-ethylamino]-ethyl)-3H-benzo-thiazol-2-one, 7-[(R)-2-(1,1-Dimethyl-2-phenyl-ethylamino)-1-hydroxy-ethyl]-4-hydroxy-3H-benzothiazol-2-one, 4-Hydroxy-7-[(R)-1-hydroxy-2-[2-(5,6,7,8-tetrahydro-naphthalen-2-yl)-ethylamino]-ethyl]-3H-benzothiazol-2-one formate, 7-[(R)-2-((1S,2S)-2-Benzyl-oxy-cyclopentyl-amino)-1-hydroxy-ethyl]-4-hydroxy-3H-benzo-thiazol-2-one and 7-[(R)-2-((1S,2R)-2-Benzyl-oxy-cyclopentylamino)-1-hydroxy-ethyl]-4-hydroxy-3H-benzothiazol-2-one respectively These compounds are prepared using the procedures described in international patent application WO Z004/016601.

### Examples 1-60

Gelatin capsules suitable for use in a capsule inhaler such as that described in U.S. Pat. No. 3,991,761 and EP 1270034 are prepared, each capsule containing a dry powder obtained by mixing Compound A1 and Compound B1 which have been ground to a mean particle diameter of 1 to 5 μm and lactose monohydrate having a particle diameter below 212 μm, the amounts being as shown in the Table 1 below:

TABLE 1

| | Compound A1 | Compound B1 | Lactose |
|---|---|---|---|
| Example | (Parts) | (Parts) | (Parts) |
| 1 | 20 | 100 | 19880 |
| 2 | 40 | 100 | 19860 |
| 3 | 80 | 100 | 19820 |
| 4 | 100 | 100 | 19800 |
| 5 | 120 | 100 | 19780 |
| 6 | 140 | 100 | 19760 |
| 7 | 160 | 100 | 19740 |
| 8 | 180 | 100 | 19720 |
| 9 | 200 | 100 | 19700 |
| 10 | 220 | 100 | 19680 |
| 11 | 240 | 100 | 19660 |
| 12 | 300 | 100 | 19600 |
| 13 | 500 | 100 | 19400 |
| 14 | 1000 | 100 | 18900 |
| 15 | 2000 | 100 | 17900 |
| 16 | 20 | 100 | 24880 |
| 17 | 40 | 100 | 24860 |
| 18 | 80 | 100 | 24820 |
| 19 | 100 | 100 | 24800 |
| 20 | 120 | 100 | 24780 |
| 21 | 140 | 100 | 24760 |
| 22 | 160 | 100 | 24740 |
| 23 | 180 | 100 | 24720 |
| 24 | 200 | 100 | 24700 |
| 25 | 220 | 100 | 24680 |
| 26 | 240 | 100 | 24660 |
| 27 | 300 | 100 | 24600 |
| 28 | 500 | 100 | 24400 |
| 29 | 1000 | 100 | 23900 |
| 30 | 2000 | 100 | 22900 |
| 31 | 20 | 200 | 14780 |
| 32 | 40 | 200 | 14760 |
| 33 | 80 | 200 | 14720 |
| 34 | 100 | 200 | 14700 |
| 35 | 120 | 200 | 14680 |
| 36 | 140 | 200 | 14660 |
| 37 | 160 | 200 | 14640 |
| 38 | 180 | 200 | 14620 |
| 39 | 200 | 200 | 14600 |
| 40 | 220 | 200 | 14580 |
| 41 | 240 | 200 | 14560 |
| 42 | 300 | 200 | 14500 |
| 43 | 500 | 200 | 14300 |
| 44 | 1000 | 200 | 13800 |
| 45 | 2000 | 200 | 12800 |
| 46 | 20 | 200 | 24780 |
| 47 | 40 | 200 | 24760 |
| 48 | 80 | 200 | 24720 |
| 49 | 100 | 200 | 24700 |
| 50 | 120 | 200 | 24680 |
| 51 | 140 | 200 | 24660 |
| 52 | 160 | 200 | 24640 |
| 53 | 180 | 200 | 24620 |
| 54 | 200 | 200 | 24600 |
| 55 | 220 | 200 | 24580 |
| 56 | 240 | 200 | 24560 |
| 57 | 300 | 200 | 24500 |
| 58 | 500 | 200 | 24300 |
| 59 | 1000 | 200 | 23800 |
| 60 | 2000 | 200 | 22800 |

### Example 61-105

A dry powder suitable for delivery from the reservoir of the multi-dose inhaler described in WO 97/20589 is prepared by mixing Compound A1 and Compound B2 which have been ground to a mean particle diameter of 1-5 μm and lactose monohydrate having a particle diameter below 212 μm, the amounts being as shown in the Table 2 below:

TABLE 2

| | Compound A1 | Compound B2 | Lactose |
|---|---|---|---|
| Example | (Parts) | (Parts) | (Parts) |
| 61 | 20 | 100 | 4880 |
| 62 | 40 | 100 | 4860 |
| 63 | 80 | 100 | 4820 |
| 64 | 100 | 100 | 4800 |
| 65 | 120 | 100 | 4780 |
| 66 | 140 | 100 | 4760 |
| 67 | 160 | 100 | 4740 |
| 68 | 180 | 100 | 4720 |
| 69 | 200 | 100 | 4700 |
| 70 | 220 | 100 | 4680 |
| 71 | 240 | 100 | 4660 |
| 72 | 300 | 100 | 4600 |
| 73 | 500 | 100 | 4400 |
| 74 | 1000 | 100 | 3900 |
| 75 | 2000 | 100 | 2900 |
| 76 | 20 | 200 | 9780 |
| 77 | 40 | 200 | 9760 |
| 78 | 80 | 200 | 9720 |
| 79 | 100 | 200 | 9700 |
| 80 | 120 | 200 | 9680 |
| 81 | 140 | 200 | 9660 |
| 82 | 160 | 200 | 9640 |
| 83 | 180 | 200 | 9620 |
| 84 | 200 | 200 | 9600 |
| 85 | 220 | 200 | 9580 |
| 86 | 240 | 200 | 9560 |
| 87 | 300 | 200 | 9500 |
| 88 | 500 | 200 | 9300 |
| 89 | 1000 | 200 | 8800 |
| 90 | 2000 | 200 | 7800 |
| 91 | 20 | 250 | 14730 |
| 92 | 40 | 250 | 14710 |
| 93 | 80 | 250 | 14670 |
| 94 | 100 | 250 | 14650 |
| 95 | 120 | 250 | 14630 |
| 96 | 140 | 250 | 14610 |
| 97 | 160 | 250 | 14590 |
| 98 | 180 | 250 | 14570 |
| 99 | 200 | 250 | 14550 |
| 100 | 220 | 250 | 14530 |
| 101 | 240 | 250 | 14510 |
| 102 | 300 | 250 | 14450 |
| 103 | 500 | 250 | 14250 |
| 104 | 1000 | 250 | 13750 |
| 105 | 2000 | 250 | 12750 |

### Example 106-150

A dry powder suitable for delivery from the reservoir of the multi-dose inhaler described in WO 97/20589 is prepared by mixing Compound A1 and Compound B3 which have been ground to a mean particle diameter of 1-5 μm and lactose monohydrate having a particle diameter below 212 μm, the amounts being as shown in the Table 2 but also containing 0.5% magnesium stearate by weight.

### Examples 151-195

A dry powder suitable for delivery from the reservoir of the multi-dose inhaler described in WO 97/20589 is prepared by mixing Compound A1 and Compound B3 which have been ground to a mean particle diameter of 1-5 μm and lactose monohydrate having a particle diameter below 212 μm, the amounts being as shown in the Table 2 but also containing 1% magnesium stearate by weight.

### Examples 196-213

Aerosol formulations are prepared by dispensing micronised active ingredients, Compound A1 and Compound B4, and if required, lactose as bulking agent into a vial, sealing the vial with a metering valve, injecting the premixed ethanol/propellant and optional surfactant into the vial through the valve and subjecting the vial to ultrasonic energy to disperse the solid particles. The components and amounts used are shown in Table 3 below:

TABLE 3

| | Cpd. A1 | Cpd. B4 | HFA134a | HFA227 | Ethanol | OA | Lactose |
|---|---|---|---|---|---|---|---|
| Ex. | (Parts) | (Parts) | (Parts) | (Parts) | (Parts) | (Parts) | (Parts) |
| 196 | 2 | 10 | 36500 | 60750 | 2500 | — | 70 |
| 197 | 4 | 10 | 3410 | 6340 | 230 | 0.3 | — |
| 198 | 8 | 10 | 97000 | — | 2500 | — | 90 |
| 199 | 10 | 10 | 30500 | 67000 | 2500 | 0.5 | 100 |
| 200 | 12 | 10 | 3150 | 6550 | 250 | 1 | — |
| 201 | 14 | 10 | 3700 | 6050 | 250 | 0.8 | — |
| 202 | 16 | 10 | 3800 | 5900 | 230 | 0.4 | — |
| 203 | 18 | 10 | 4700 | 5050 | 250 | 1 | — |
| 204 | 20 | 20 | 3600 | 6150 | 225 | 1 | — |
| 205 | 22 | 20 | 3500 | 6200 | 230 | 1 | — |
| 206 | 24 | 20 | 98000 | — | 2500 | 1 | — |
| 207 | 30 | 20 | 3900 | 5900 | 250 | 1 | — |
| 208 | 2 | 20 | 30000 | 67000 | 2250 | 0.2 | 90 |
| 209 | 10 | 20 | 3500 | 6200 | 250 | 0.5 | — |
| 210 | 14 | 20 | 3200 | 6500 | 230 | 1 | — |
| 211 | 18 | 20 | 3100 | 6200 | 225 | 0.8 | — |
| 212 | 20 | 20 | 3150 | 6100 | 225 | 1 | — |
| 213 | 24 | 20 | 30000 | 60000 | 2000 | 0.8 | — |

### Examples 314-223

Aerosol formulations are prepared by dispensing micronised active ingredients, Compound A1 and Compound B5, and if required, lactose as bulking agent into a vial, sealing the vial with a metering valve, injecting the premixed ethanol/propellant and optional surfactant into the vial through the valve and subjecting the vial to ultrasonic energy to disperse the solid particles. The components and amounts used are shown in Table 4 below:

TABLE 4

| | Cpd. A1 | Cpd. B5 | HFA134a | HFA227 | Ethanol | OA | Lactose |
|---|---|---|---|---|---|---|---|
| Ex. | (Parts) | (Parts) | (Parts) | (Parts) | (Parts) | (Parts) | (Parts) |
| 214 | 4 | 10 | 34000 | 53000 | 2250 | 0.3 | 50 |
| 215 | 8 | 10 | 92000 | — | 2500 | 0.5 | 70 |
| 216 | 12 | 10 | 3000 | 5500 | 200 | — | — |
| 217 | 16 | 10 | 2500 | 5000 | 200 | 0.3 | — |
| 218 | 20 | 10 | 2000 | 3000 | 150 | 0.2 | — |
| 219 | 30 | 10 | 2000 | 2000 | 150 | 0.2 | — |
| 220 | 8 | 20 | 20000 | 25000 | 1500 | 0.2 | — |
| 221 | 12 | 20 | 2500 | 2500 | 200 | 0.2 | — |
| 222 | 20 | 20 | 2000 | 2000 | 150 | 0.2 | — |
| 223 | 30 | 20 | 20000 | 20000 | 1500 | 0.2 | — |

### Examples 224-233

The procedure of Examples 214-223 is repeated, but replacing Compound B5 with Compound B6, using amounts as shown in Table 4 above.

1. A pharmaceutical composition comprising:
(A) glycopyrrolate;
(B) (R)-5-[2-(5,6-diethyl-indan-2-ylamino)-1-hydroxyethyl]-8-hydroxy-1H-quinolin-2-one in free or salt form; and
(C) a pharmaceutically acceptable carrier.

2. A composition according to claim 1 wherein (A) is a racemate.

3. A composition according to claim 2 wherein (A) is a racemate of (3S,2′R)-3-[(cyclopentyl-hydroxyphenylacetyl)oxy]-1,1-dimethylpyrrolidinium bromide and (2S,3′R)-3[(cyclopentyl-hydroxyphenylacetyl)oxy]-1,1-dimethylpyrrolidinium bromide.

4. A composition according to claim 1, wherein (B) is (R)-5-[2-(5,6-diethyl-indan-2-ylamino)-1-hydroxyethyl]-8-hydroxy-1H-quinolin-2-one maleate.

5. A composite according to claim 1, wherein (B) is (R)-5-[2-(5,6-diethyl-indan-2-ylamino)-1-hydroxyethyl]-8-hydroxy-1H-quinolin-2-one acetate.

6. A composition according to claim 1, wherein the molar ratio of (A) to (B) is from 5:1 to 1:10.

7. A composition according to claim 6, wherein the molar ratio of (A) to (B) is from 3:1 to 1:7.

8. A composition according to claim 7, wherein the molar ratio of (A) to (B) is from 2:1 to 1:2.

9. A composition according to claim 1, wherein (C) is lactose.

10. A composition according to claim 9, wherein (C) is lactose monohydrate.

11. A composition according to claim 1 that further comprises magnesium stearate.
